(19) United States
(12) Patent Application Publication
(10) Pub. No.: US 2022/0378533 A1
(43) Pub. Date: Dec. 1, 2022

(54) CONTROL DRIVE ASSEMBLIES FOR ROBOTIC SURGICAL SYSTEMS

(71) Applicant: Titan Medical, Inc., Toronto (CA)

(72) Inventors: Ian McDiarmid, Worthing (GB); Brian King, Stowmarket (GB); Jon Cooke, Cambridge (GB); Aki Hannu Einari Laakso, Raleigh, NC (US); Hans Christian Pflaumer, Apex, NC (US); Akshaya Ahuja, St. Neots (GB); Chace F. Medeiros, Raleigh, NC (US)

(21) Appl. No.: 17/686,890

(22) Filed: Mar. 4, 2022

Related U.S. Application Data
(60) Provisional application No. 63/193,750, filed on May 27, 2021.

(51) Int. Cl. A61B 34/35; A61B 34/00; A61B 34/37; A61B 90/50

(52) U.S. Cl. CPC A61B 34/35; A61B 34/71; A61B 34/37; A61B 34/25; A61B 90/50; A61B 2034/301

(57) ABSTRACT

A control unit assembly for a robotic surgical system includes a housing, a surgical instrument, and a motor block assembly supported within the housing. The housing includes a front face, a rear face, and a sidewall extending between the front and rear faces. The motor block assembly includes a motor block module that is selectively attachable to the surgical instrument. The motor block module is remotely actuatable to control the surgical instrument. The motor block module is axially movable relative to the housing to facilitate movement of the surgical instrument relative to the housing.

### CROSS REFERENCE TO RELATED APPLICATIONS

This application claims the benefit of U.S. Provisional Application Ser. No. 63/193,750, filed May 27, 2021, the entire contents of which are incorporated by reference herein.

### TECHNICAL FIELD

This disclosure relates to robotic systems and, more particularly, to control drive assemblies for robotic surgical systems.

### BACKGROUND

Robotic surgical systems include control drive assemblies supporting surgical instruments used in laparoscopic and/or robotic surgery. These surgical instruments generally have a proximally located actuating mechanism that is operably coupled to a control drive unit of a control drive assembly for actuating distal end effectors of the surgical instruments. The control drive unit includes any number of motors operably associated with the actuating mechanisms of the surgical instruments. These motors are remotely controlled by a clinician to enable the surgical instruments to robotically perform a surgical task within a body cavity of a patient, and often in remote locations within the body cavity that are not easily accessed without robotic surgical systems.

### SUMMARY

According to an aspect of this disclosure, a robotic surgical system includes an instrument cart having a setup arm assembly and a control drive assembly coupled to the instrument cart. The control drive assembly includes at least one surgical instrument and a control drive unit. The control drive unit is secured to the setup arm assembly. The control drive unit is movable relative to the setup arm assembly. The control drive unit includes a housing and a motor block assembly supported within the housing. The housing includes a front face, a rear face, and a sidewall extending between the front and rear faces. The motor block assembly includes at least one motor block module that is selectively attachable to the at least one surgical instrument via front or side loading. The at least one motor block module is remotely actuatable to control the at least one surgical instrument. The at least one motor block module is axially movable relative to the housing to facilitate movement of the at least one surgical instrument relative to the housing.

In aspects, the control drive unit may include a mounting clevis that secures the housing to the setup arm assembly. The mounting clevis may be pinned to the sidewall of the housing to enable the housing to pitch relative to the mounting clevis. The mounting clevis may be secured to the setup arm assembly by a yaw pin that enables the housing to yaw with the mounting clevis relative to the setup arm assembly.

In aspects, the at least one motor block module may include a plurality of motor block modules. Each motor block module of the plurality of motor block modules may be independently axially movable relative to the other motor block modules.

In aspects, the robotic surgical system may further include a support arm extending from the control drive unit. The support arm may be secured to an insertion tube on a distal end portion of support arm.

In aspects, the at least one surgical instrument may include a plurality of surgical instruments, and wherein the plurality of surgical instruments may be secured to the plurality of motor block modules.

In aspects, the plurality of surgical instruments may be selectively movable through the insertion tube.

In aspects, the at least one motor block module may support a sterile interface module that couples the at least one surgical instrument to the at least one motor block module.

In aspects, the at least one motor block module may include at least one motor that imparts rotational force to the at least one surgical instrument.

According to another aspect, this disclosure is directed to a surgical system. The surgical system includes a housing, a support arm, an insertion tube, at least one surgical instrument, a motor block assembly. The housing includes a front face, a rear face, and a sidewall extending between the front and rear faces. The support arm extends distally from the front face of the housing. The insertion tube is supported on a distal end portion of the support arm. The motor block assembly is supported within the housing. The motor block assembly includes at least one motor block module that is selectively attachable to the at least one surgical instrument via front or side loading. The at least one motor block module is remotely actuatable to control the at least one surgical instrument. The at least one motor block module is axially movable relative to the housing to facilitate movement of the at least one surgical instrument relative to the insertion tube.

In aspects, the housing may support a mounting clevis for selectively securing the housing to an instrument cart. The mounting clevis may be pinned to the sidewall of the housing to enable the housing to pitch relative to the mounting clevis. The mounting clevis may be selectively securable to a setup arm assembly of the instrument cart by a yaw pin that enables the housing to yaw with the mounting clevis relative to the setup arm assembly.

In aspects, the at least one motor block module may include a plurality of motor block modules. Each motor block module of the plurality of motor block modules may be independently axially movable relative to the other motor block modules. The at least one surgical instrument may include a plurality of surgical instruments. The plurality of surgical instruments may be secured to the plurality of motor block modules. The plurality of surgical instruments may be selectively movable through the insertion tube.

In aspects, the at least one motor block module may support a sterile interface module that couples the at least one surgical instrument to the at least one motor block module.

In aspects, the at least one motor block module may include at least one motor that imparts rotational force to the at least one surgical instrument.

According to yet another aspect of this disclosure, a control unit assembly for a robotic surgical system includes a housing, at least one surgical instrument, and a motor block assembly. The housing includes a front face, a rear face, and a sidewall extending between the front and rear faces. The motor block assembly is supported within the housing. The motor block assembly includes at least one motor block module that is selectively attachable to the at least one surgical instrument via front or side loading. The at least one motor block module is remotely actuatable to control the at least one surgical instrument. The at least one motor block module is axially movable relative to the housing to facilitate movement of the at least one surgical instrument relative to the housing.

According to another aspect, a robotic surgical system includes an instrument cart and a control drive assembly. The instrument cart has a setup arm assembly. The control drive assembly is coupled to the instrument cart. The control drive assembly includes at least one surgical instrument and a control drive unit. The control drive unit is secured to the setup arm assembly. The control drive unit is movable relative to the setup arm assembly. The control drive unit includes a housing and a motor block assembly supported within the housing. The housing includes a front face, a rear face, and a sidewall extending between the front and rear faces. The motor block assembly includes at least one motor block module that is selectively attachable to the at least one surgical instrument when the at least one surgical instrument is loaded into the housing through the rear face of the housing. The at least one motor block module is remotely actuatable to control the at least one surgical instrument. The at least one motor block module is axially movable relative to the housing to facilitate movement of the at least one surgical instrument relative to the housing.

In aspects, a proximal portion of the at least one surgical instrument may laterally engage with the at least one motor block module to impart rotational force from the at least one motor block module to the proximal portion of the at least one surgical instrument.

In aspects, the at least one motor block module may include at least one motor.

According to yet another aspect, this disclosure is directed to a surgical system. The surgical system includes a housing, a support arm, an insertion tube, at least one surgical instrument and a motor block assembly. The housing includes a front face, a rear face, and a sidewall extending between the front and rear faces. The support arm extends distally from the front face of the housing. The insertion tube is supported on a distal end portion of the support arm. The motor block assembly is supported within the housing. The motor block assembly includes at least one motor block module that is selectively attachable to the at least one surgical instrument when the at least one surgical instrument is loaded into the housing through the rear face of the housing. The at least one motor block module is remotely actuatable to control the at least one surgical instrument. The at least one motor block module is axially movable relative to the housing to facilitate movement of the at least one surgical instrument relative to the insertion tube.

According to a further aspect, this disclosure is directed to a control unit assembly for a robotic surgical system. The control unit assembly includes a housing, at least one surgical instrument, and a motor block assembly. The housing includes a front face, a rear face, and a sidewall extending between the front and rear faces. The motor block assembly is supported within the housing. The motor block assembly includes at least one motor block module that is selectively attachable to the at least one surgical instrument when the at least one surgical instrument is loaded into the housing through the rear face of the housing. The at least one motor block module is remotely actuatable to control the at least one surgical instrument. The at least one motor block module is axially movable relative to the housing to facilitate movement of the at least one surgical instrument relative to the housing.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate aspects of this disclosure and, together with a general description of this disclosure given above, and the detailed description given below, explain the principles of this disclosure, wherein:

FIG. 1 is a perspective view of a robotic surgical system being used for a surgical procedure on a patient in accordance with the principles of this disclosure;

FIGS. 2-4 are progressive views illustrating surgical instruments of the robotic surgical system of FIG. 1 being manipulated within a body cavity of the patient;

FIG. 5 is an enlarged, perspective view of the robotic surgical system of FIG. 1 and illustrating a control drive assembly thereof being used to perform a surgical procedure on a patient;

FIG. 6 is an enlarged, perspective view of a control drive unit of the control drive assembly of FIG. 5;

FIG. 7 is a perspective view of the control drive unit of FIG. 6 with surgical instruments shown attached thereto and disposed in a retracted position;

FIG. 8 is a perspective view of FIG. 7 with one of the surgical instruments shown in an extended position;

FIG. 9 is a perspective view of FIG. 7 with the surgical instruments shown in extended positions;

FIG. 10 is an enlarged, perspective view the robotic surgical system of FIG. 1 illustrating another control drive assembly thereof being used to perform a surgical procedure on a patient;

FIG. 11 is an enlarged, perspective view of a control drive unit of the control drive assembly of FIG. 10;

FIGS. 12-15 are progressive views illustrating surgical instruments being attached to the control drive unit of FIG. 11;

FIG. 16 is a front, perspective view illustrating one of the surgical instruments in a retracted position and other surgical instruments in extended positions;

FIG. 17 is a rear, perspective view of FIG. 16;

FIG. 18 is a front, perspective view of another control drive assembly in accordance with the principles of this disclosure;

FIG. 19 is a rear view of the control drive assembly of FIG. 18;

FIG. 20 is a front, perspective view of still another control drive assembly in accordance with the principles of this disclosure;

FIG. 21 is a rear view of the control drive assembly of FIG. 20;

FIG. 22 is a perspective view of yet another control drive assembly in accordance with the principles of this disclosure, the surgical instruments thereof shown in extended positions;

FIG. 23 is a perspective view of one control drive assembly in accordance with the principles of this disclosure, the control drive assembly shown with one surgical instrument attached thereto and disposed in a retracted position; and

FIG. 24 is a perspective view of another control drive assembly in accordance with the principles of this disclosure, the surgical instruments thereof shown in retracted positions.

### DETAILED DESCRIPTION

Aspects of this disclosure are described in detail with reference to the drawings, in which like reference numerals designate identical or corresponding elements in each of the several views. As used herein, the term “distal” refers to that portion of structure farther from the user, while the term “proximal” refers to that portion of structure, closer to the user. As used herein, the term “clinician” refers to a doctor, nurse, or other care provider and may include support personnel and/or equipment operators.

In the following description, well-known functions or constructions are not described in detail to avoid obscuring the present disclosure in unnecessary detail.

Robotic surgical systems have been used in minimally invasive medical procedures and can include robotic arm assemblies. Such procedures may be referred to as what is commonly referred to as “Telesurgery.” Some robotic arm assemblies include one or more robot arms to which surgical instruments can be coupled. Such surgical instruments include, for example, endoscopes, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments.

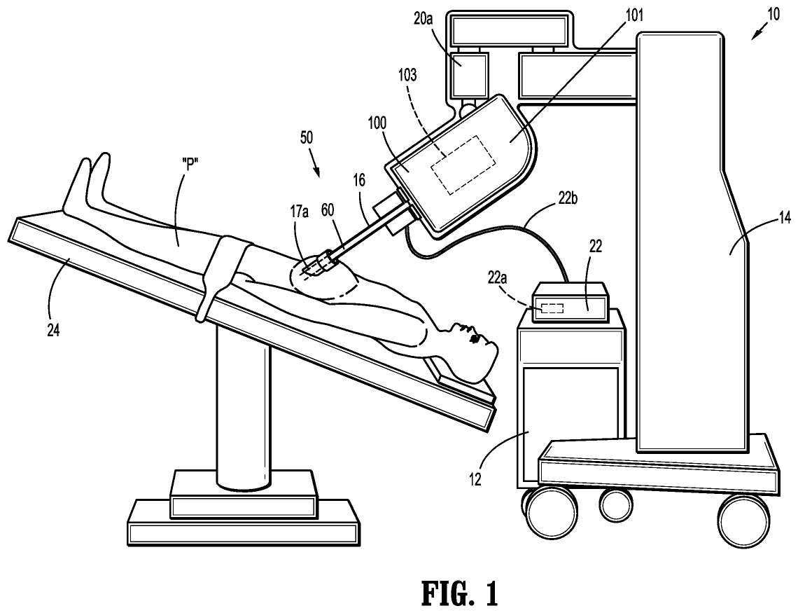

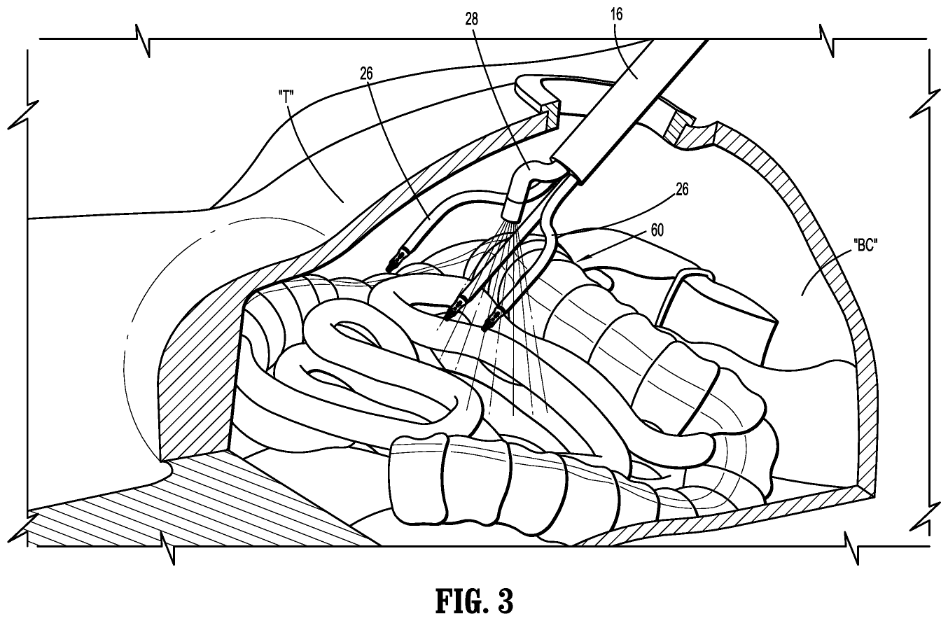

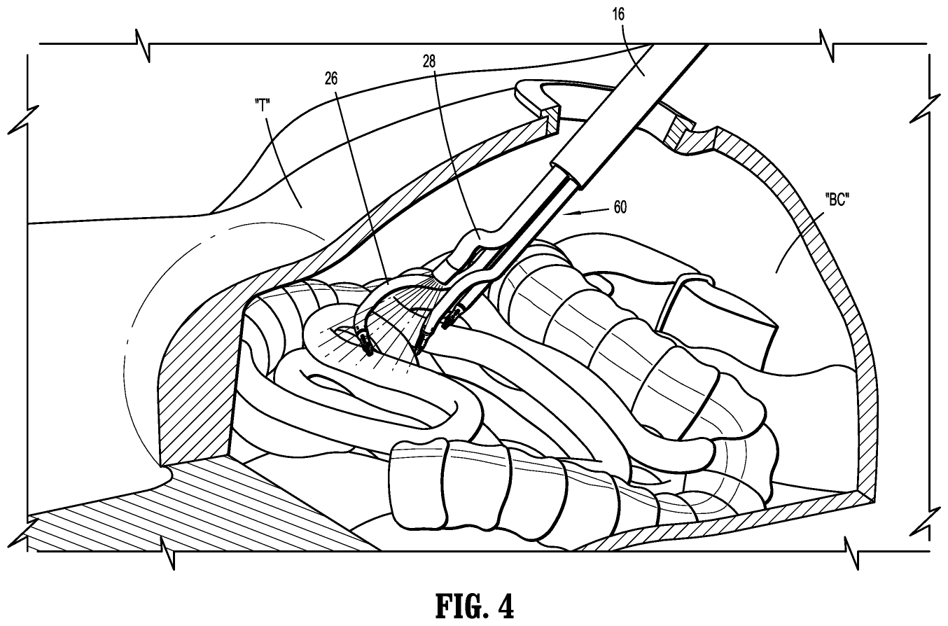

With reference to FIGS. 1-5, a robotic surgical system is shown generally at 10. Robotic surgical system 10 employs various robotic elements to assist the clinician and allow remote operation (or partial remote operation) of surgical instruments 60 of surgical instrument systems 50 of robotic surgical system 10. Various controllers, circuitry, robotic arms, gears, cams, pulleys, electric and mechanical motors, etc. may be employed for this purpose and may be designed with surgical system 10 to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a workstation 12 and an instrument cart 14. The instrument cart 14 supports a control drive assembly 100. Control drive assembly 100 includes one or more surgical instrument systems 50 mounted on a control drive unit 101. Control drive unit 101 is movable relative to cart 14 and houses an instrument drive assembly 103 for manipulating the surgical instrument systems 50 and/or independent surgical instruments 60 thereof with the assistance of, for example one or more computing devices or controllers. Although only four surgical instruments 60 are shown, surgical instrument system 50 can include any number and/or type of surgical instruments. The surgical instruments 60 can include, for example, graspers or forceps 26, which may be electrosurgical, an endoscope 28, and/or any other suitable instrument that can be driven by one or more associated tool drives (not shown) of instrument drive assembly 103. For example, besides graspers 26 and endoscope 28, the one or more surgical instruments 60 can include dexterous tools, such as grippers, needle drivers, staplers, dissectors, cutters, hooks, graspers, scissors, coagulators, irrigators, suction devices, that are used for performing a surgical procedure.

Surgical instrument system 50 includes an insertion tube 16 defining a plurality of separate conduits, channels or lumens 16a therethrough that are configured to receive, for instance, the surgical instruments 60 for accessing a body cavity “BC” of a patient “P.” In other aspects, the insertion tube 16 may define a single conduit, channel or lumen therethrough that is configured to receive, for instance, the surgical instruments 60 for accessing a body cavity “BC” of a patient “P.” In particular, the insertion tube 16 can be inserted through an incision “I” and/or access devices 17a, 17b (e.g., a surgical portal, which may include or more seals to facilitate sealed insertion through tissue “T” of the patient “P”) and into the body cavity “BC” of the patient “P”). With insertion tube 16 positioned in the patient “P,” the surgical instruments 60 can be advanced through insertion tube 16 into the body cavity “BC” of the patient “P.” Further, the workstation 12 includes an input device 22 in communication with control drive unit 101 for use by a clinician to control the insertion tube 16 and the various surgical instrument systems 50 (and surgical instruments 60 thereof) via the instrument drive assembly 103 for performing surgical operations on the patient “P” while the patient “P” is supported on a surgical table 24, for example. Input device 22 is configured to receive input from the clinician and produces input signals. Input device 22 may also be configured to generate feedback to the clinician. The feedback can be visual, auditory, haptic, or the like.

The workstation 12 can further include computing devices and/or controllers such as a master processor circuit 22a in communication with the input device 22 for receiving the input signals and generating control signals for controlling the robotic surgical system 10, which can be transmitted to the instrument cart 14 via an interface cable 22b. In some cases, transmission can be wireless and interface cable 22b may not be present. The input device 22 can include right and left-hand controls (not shown) and/or foot pedals (not shown), which are moved/operated to produce input signals at the input device 22 and/or to control robotic surgical system 10. The instrument cart 14 can include a slave processor circuit 20a that receives and the control signals from the master processor circuit 22a and produces slave control signals operable to control the various surgical instrument systems 50 (and surgical instruments 60 thereof) during a surgical procedure. The workstation 12 can also include a user interface, such as a display (not shown) in communication with the master processor circuit 22a for displaying information (such as, body cavity images) for a region or site of interest (for example, a surgical site, a body cavity, or the like) and other information to a clinician. While both master and slave processor circuits are illustrated, in other aspects, a single processor circuit may be used to perform both master and slave functions.

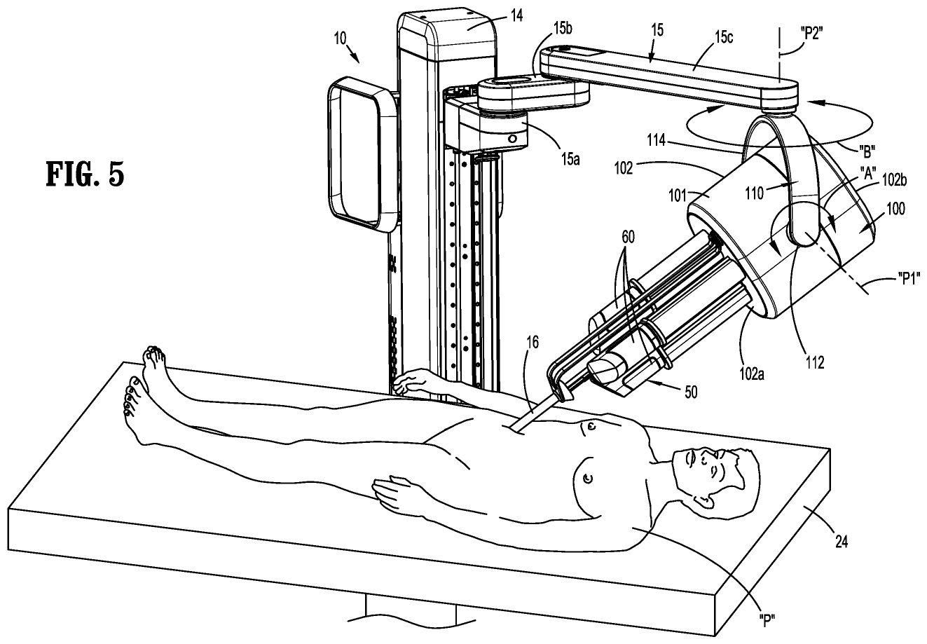

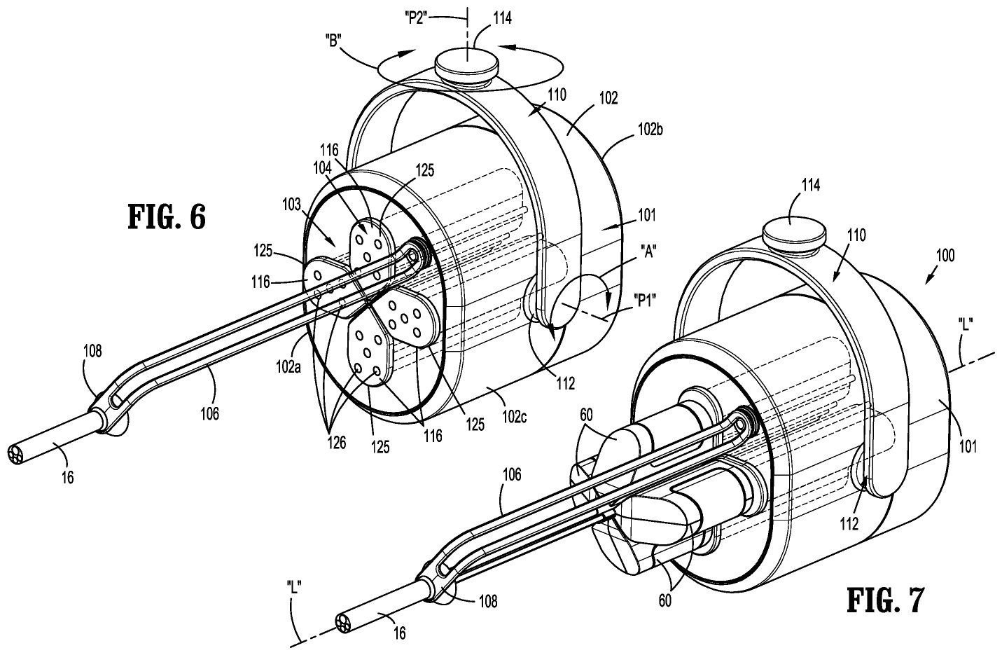

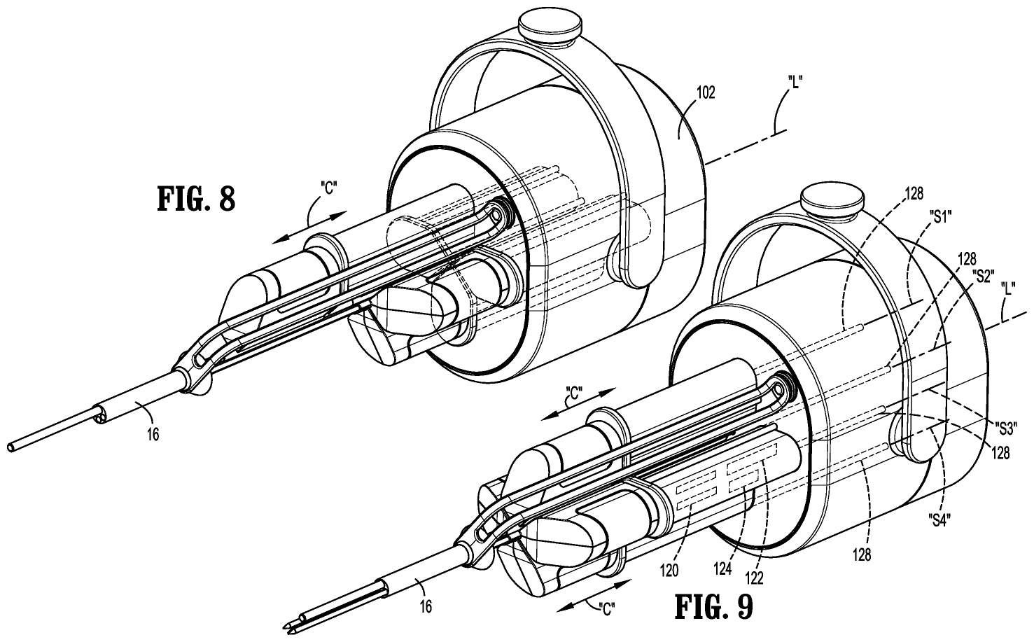

Turning now to FIGS. 5-9, control drive unit 101 of control drive assembly 100 includes a housing 102 that supports instrument drive assembly 103, a motor block assembly 104 of instrument drive assembly 103, and a support arm 106 that extends distally from housing 102 to a port latch 108 on a distal end of support arm 106 for supporting insertion tube 16.

Housing 102 of control drive unit 101 includes a front face 102a, a rear face 102b, and a sidewall 102c extending between the front and rear faces 102a, 102b. Housing 102 further includes a mounting clevis 110 that is pinned to opposite sides of sidewall 102c of housing 102 via pitch pins 112 extending from sidewall 102c to enable housing 102 to rotate (e.g., pitch in clockwise and/or counterclockwise directions such as in upward and/or downward directions) relative to mounting clevis 110 about pitch pivot axis “P1” defined through pitch pins 112, as indicated by arrows “A.” Mounting clevis 110 further includes a yaw pin 114 extending from a central portion of mounting clevis 110 that secures to a setup arm assembly 15 (FIG. 5) of robotic surgical system 10, namely, a distal setup arm 15c, of setup arm assembly 15. Distal setup arm 15c is pivotably cantilevered from an intermediate set up arm 15b, which is pivotally cantilevered from a proximal set up arm 15a that is secured to instrument cart 14. Yaw pin 114 of mounting clevis 110 enables housing 102 to rotate (e.g., yaw in clockwise and/or counterclockwise directions such as in left and/or right directions) relative to setup arm assembly 15, and with mounting clevis 110, about yaw pivot axis “P2” defined through yaw pin 114, as indicated by arrows “B.”

Motor block assembly 104 of instrument drive assembly 103 includes a plurality of motor block modules 116, each of which is independently axially movable relative to the other motor block modules 116 along a longitudinal axis “L” defined through control drive unit 101, as indicated by arrows “C.” Each motor block module 116 includes a module casing 118 that housing any number of motors 120, driver boards 122 (e.g., a controller or printed circuit board), and cooling fans 124 (for cooling motors 120 and/or driver boards 122) therein for efficiently and effectively imparting rotational driving force to surgical instruments 60 attached to the respective motor block module 116. Motors 120, driver boards 122, and/or cooling fans 124 may be disposed in electrical communication with workstation 12 to enable a clinician to operate the motor block modules 116 of motor block assembly 104. Motors 120 are coupled to instrument couplers 126 of a sterile interface module 125 supported on a distal end portion of each motor block module 116. Instrument couplers 126 are positioned to operatively engage with counterpart couplers (not shown) extending from a proximal end portion (e.g., a proximal housing portion) of surgical instruments 60 when respective surgical instruments 60 are front (proximally) and/or side (e.g., laterally or radially) loaded onto each motor block module 116 so that the rotational drive force can be transferred distally from the respective motor block module 116 to the respective surgical instrument 60.

Each motor block module 116 of motor block assembly 104 is coupled to a lead screw assembly 128 supported in housing 102 that is rotatable about respective central screw axes “S1”-“S4” thereof to axially advance the respective motor block modules 116 (with and/or relative to one another and relative to housing 102), as indicated by arrows “C.” Axial movement of motor block modules 116 relative to housing 102 enables surgical instruments 60 to be advanced axially through and/or relative to insertion tube 16 for accessing different locations with the body cavity “BC” of the patient “P” during a surgical task (see FIGS. 1-5). Also, although not specifically shown, instrument drive assembly 103 and/or motor block assembly 104 may include any number and/or type of sensors (e.g., encoders or the like) to determine rotational and/or axial positioning of one or more components of motor block assembly 104 and/or surgical instruments 60 relative to housing 102, other surgical instruments 60, insertion tube 16, support arm 106, setup arm 15, and/or patient “P”, etc.

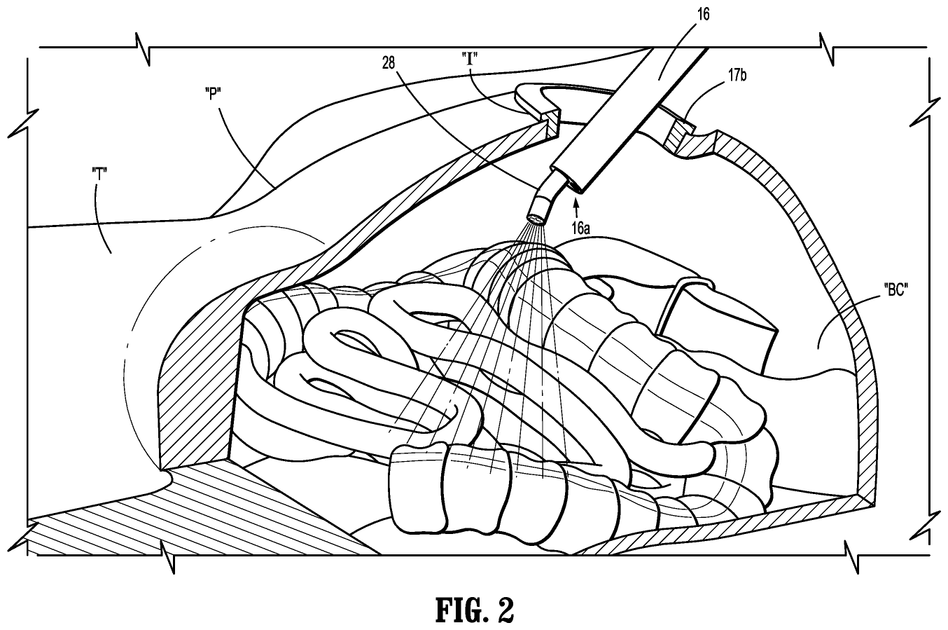

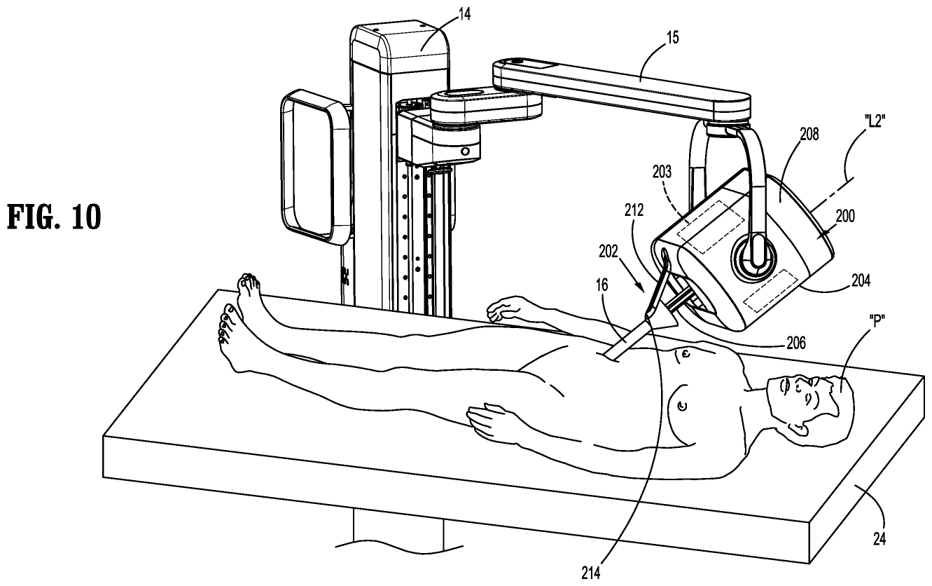

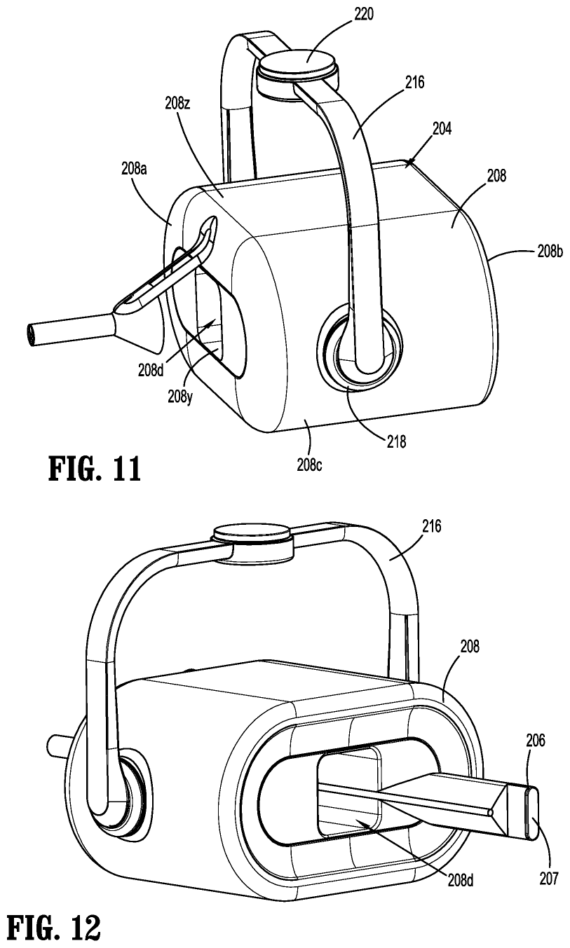

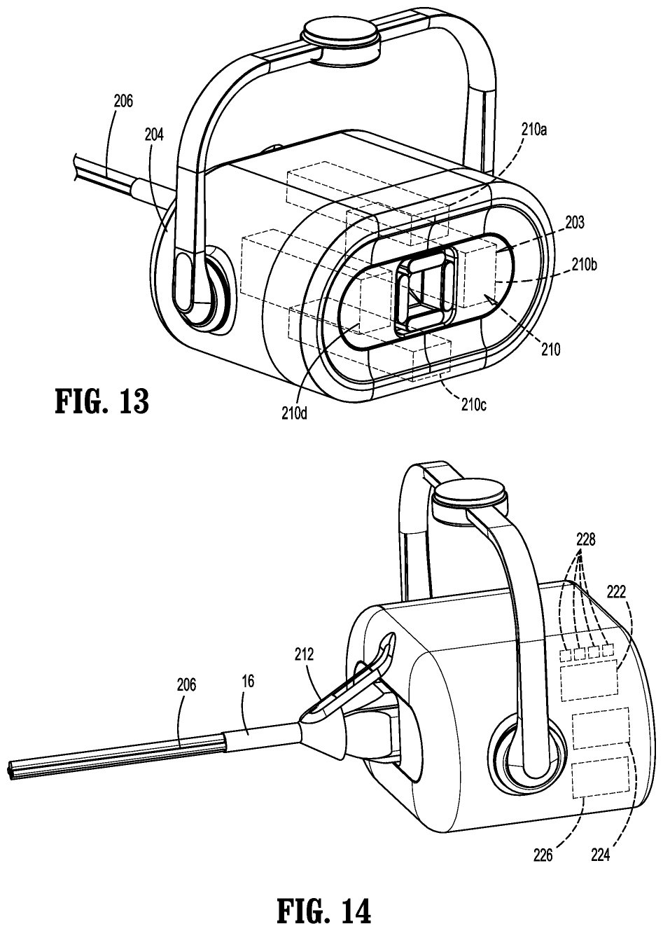

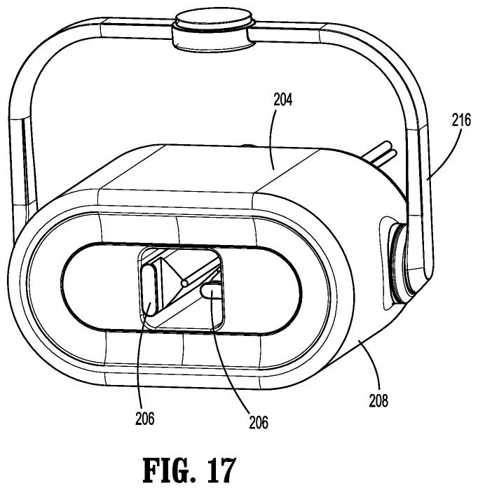

With reference to FIGS. 10-17, instrument cart 14 can support a control drive assembly 200. Control drive assembly 200 includes one or more surgical instrument systems 202 mounted on a control drive unit 204 via rear-loading, but control drive unit 204 is otherwise substantially similar to control drive unit 101. In particular, control drive unit 204 is movable relative to cart 14 and houses an instrument drive assembly 203 for manipulating the surgical instrument systems 202 and/or independent surgical instruments 206 thereof with the assistance of, for example one or more computing devices or controllers. Surgical instrument systems 202 can include insertion tube 16 for accessing a body cavity “BC” (FIG. 2) of a patient “P” and any number and/or type of surgical instruments 206 advanceable through insertion tube 16. For example, such surgical instruments 206 can include dexterous tools such as graspers, endoscopes, needle drivers, staplers, dissectors, cutters, hooks, scissors, coagulators, irrigators, suction devices etc., and/or any other suitable instrument that can be driven by one or more associated tool drives (not shown) of instrument drive assembly 203 and used for performing a surgical procedure in the body cavity “BC.” Surgical instruments 206 include a proximal housing portion 207 having a flat or paddle-shaped configuration.

Control drive unit 204 of control drive assembly 200 includes housing 208 that supports instrument drive assembly 203, a motor block assembly 210 of instrument drive assembly 203, and a support arm 212 that extends distally from housing 208 to a port latch 214 on a distal end of support arm 212 for supporting insertion tube 16.

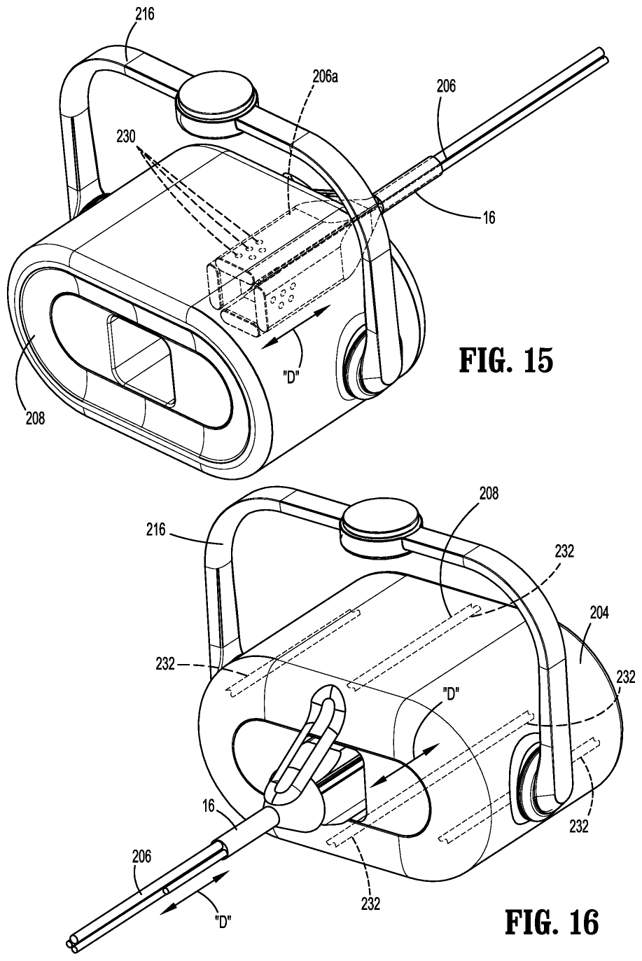

Housing 208 of control drive unit 204 includes an outer surface 208z having a front face 208a, a rear face 208b, and a sidewall 208c extending between the front and rear faces 208a, 208b. Housing 208 further includes an inner surface 208y defining an elongated instrument channel 208d through housing 208. Elongated instrument channel 208d is positioned to slidably receive surgical instruments 206 therein for enabling drive assemblies (see e.g., drive assemblies 230 shown in FIG. 15) of proximal housing portions of surgical instruments 206 to radially and/or laterally engage motor block modules 210a, 210b, 210c, 210d of motor block assembly 210 of instrument drive assembly 203 when surgical instruments 206 are rear-loaded into control drive unit 204. Once surgical instruments 206 are rear-loaded, surgical instruments 206 are positioned to axially advance relative to housing 208 with the respective motor block modules 210a, 210b, 210c, 210d of motor block assembly 310.

Housing 208 of control drive unit 204 further includes a mounting clevis 216 that is pinned to opposite sides of sidewall 208c of housing 208 via pitch pins 218 extending from sidewall 208c to enable housing 102 to rotate (e.g., pitch in clockwise and/or counterclockwise directions such as in upward and/or downward directions) relative to mounting clevis 216. Mounting clevis 216 further includes a yaw pin 220 extending from a central portion of mounting clevis 216 that secures to setup arm assembly 15 and enables housing 208 to rotate (e.g., yaw in clockwise and/or counterclockwise directions such as in left and/or right directions) relative to setup arm assembly 15.

Motor block assembly 210 of instrument drive assembly 203 includes motor block modules 210a, 210b, 210c, 210d, each of which is independently axially movable relative to and/or with the other motor block modules along a longitudinal axis “L2” defined through control drive unit 204. Each motor block module 210a, 210b, 210c, 210d includes any number of motors 222, driver boards 224 (e.g., a controller or printed circuit board), and cooling fans 226 (for cooling motors 222 and/or driver boards 224) therein for efficiently and effectively imparting rotational driving force to surgical instruments 206 attached to the respective motor block module 210a, 210b, 210c, 210d. Motors 222, driver boards 224, and/or cooling fans 226 may be disposed in electrical communication with workstation 12 to enable a clinician to operate the motor block modules 210a, 210b, 210c, 210d of motor block assembly 210. Motors 222 are coupled to instrument couplers 228 supported on each motor block module 210a, 210b, 210c, 210d and disposed in registration with elongated instrument channel 208d.

Instrument couplers 228 of motor block assembly 210 are positioned to operatively engage with driving assemblies 230 having counterpart couplers extending laterally from a sidewall 206a of a proximal end portion of surgical instruments 206 when respective surgical instruments 206 are rear-loaded into control drive unit 204. Notably, such driving assemblies 230 can include any number and/or arrangement of cranks, pulleys, sliders, cables, racks, pinions, gears, etc. to facilitate operation of a distal end effector of such surgical instruments 206 (e.g., graspers, staplers, shears, endoscopes, etc.). Each motor block module 210a, 210b, 210c, 210d is positioned so that the rotational drive force can be transferred from the respective motor block module 116, through instrument couplers 228, to the respective surgical instrument 206 via a side or lateral drive.

To effectuate the axial movement of each motor block module 210a, 210b, 210c, 210d of motor block assembly 210, each motor block module 210a, 210b, 210c, 210d is coupled to a lead screw assembly 232 supported in housing 102 that is rotatable to axially advance the respective motor block modules 210a, 210b, 210c, 210d (with and/or relative to one another and relative to housing 208), as indicated by arrows “D.” Axial movement of motor block modules 210a, 210b, 210c, 210d relative to housing 208 enables surgical instruments 206 to be advanced axially through and/or relative to insertion tube 16 for accessing different locations with the body cavity “BC” of the patient “P” during a surgical task (see FIGS. 1-5). Also, although not specifically shown, instrument drive assembly 203 and/or motor block assembly 210 may include any number and/or type of sensors (e.g., encoders or the like) to determine rotational and/or axial positioning of one or more components of motor block assembly 210 and/or surgical instruments 206 relative to housing 208, other surgical instruments 206, insertion tube 16, support arm 106, setup arm 15, and/or patient “P”, etc.

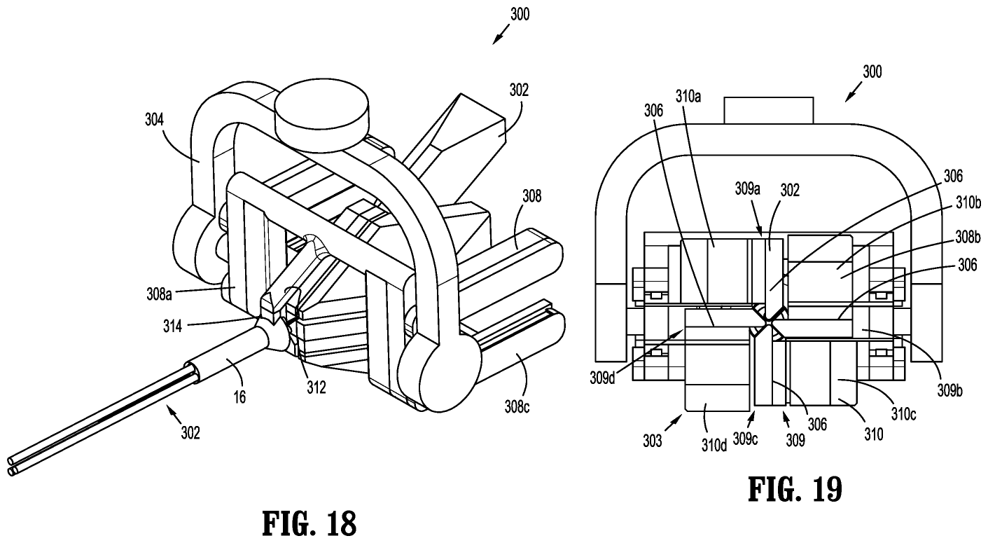

With reference to FIGS. 18 and 19, another control drive assembly is generally shown as 300. Control drive assembly 300 is similar to control drive assembly 200. In particular, control drive assembly 300 includes one or more surgical instrument systems 302 mounted on a control drive unit 304 via rear-loading.

Control drive unit 304 of control drive assembly 300 includes housing 308 that supports instrument drive assembly 203, a motor block assembly 310 of an instrument drive assembly 303, and a support arm 312 that extends distally from housing 308 to a port latch 314 on a distal end of support arm 312 for supporting insertion tube 16.

Housing 308 of control drive unit 304 includes a front portion 308a, a rear portion 308b, and a side portion 308c extending between the front and rear portions 308a, 308b. Housing 208 defines an instrument channel 309 centrally therethrough that is configured to receive surgical instruments 306 therein for respective engagement with motor block modules 310a, 310b, 310c, 310d of motor block assembly 310 disposed between adjacent surgical instruments 306. Instrument channel 309 includes channel segments 309a, 309b, 309c, 309d that are arranged in a cross or intersecting configuration (e.g., x, t, etc.). Instrument channel 309 is positioned to slidably receive surgical instruments 306 therein for enabling proximal housing portions of surgical instruments 306 to radially and/or laterally engage motor block modules 310a, 310b, 310c, 310d of motor block assembly 310 of instrument drive assembly 303 when surgical instruments 306 are rear-loaded into control drive unit 304. Once surgical instruments 306 are rear-loaded, surgical instruments 306 are positioned to axially advance relative to housing 308 with the respective motor block modules 310a, 310b, 310c, 310d of motor block assembly 310, similar to that detailed above with respect to control drive assembly 200.

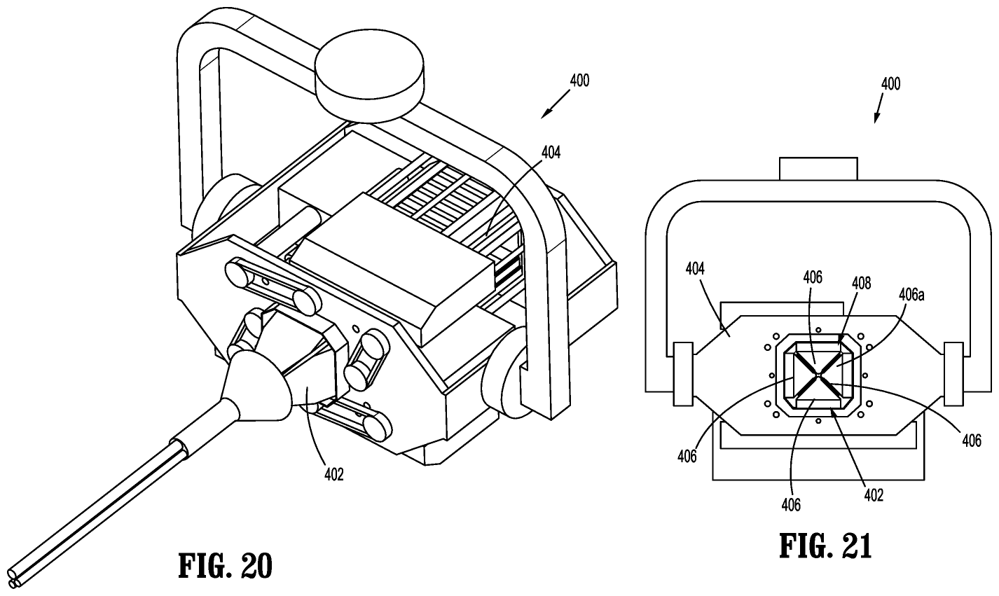

As seen in FIGS. 20 and 21, yet another control drive assembly is generally shown as 400. Control drive assembly 400 is similar to control drive assembly 300 and includes one or more surgical instrument systems 402 mounted on a control drive unit 404 via rear-loading. Instead of the cross or intersecting configuration illustrated in FIGS. 18 and 19 with respect to control drive assembly 300, control drive assembly 400, namely, control drive unit 404 and surgical instruments 406 of surgical instruments systems 402, are arranged to support surgical instruments 406 in a square configuration within a square-shaped instrument channel 408 defined through control drive unit 404. Each surgical instrument 406 has a proximal housing 406a that has a triangular configuration.

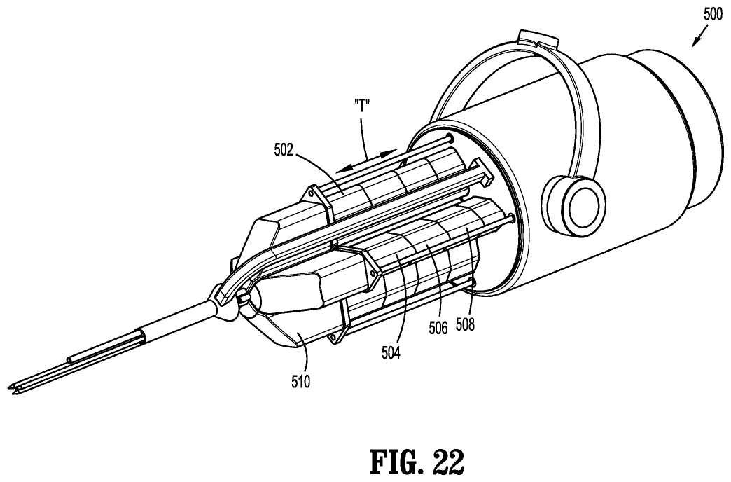

With reference to FIG. 22, still another control drive assembly is generally shown as 500. Control drive assembly 500 is similar to control drive assembly 100, but includes motor block modules 502 having a number of telescopic segments 504, 506, 508, etc. that are movable relative to one another (and may be positioned to nest within one another) to move each motor block module 502 between extended and retracted positions, as indicated by arrows “T,” for axially retracting or advancing surgical instruments 510 secured to distal end portions thereof.

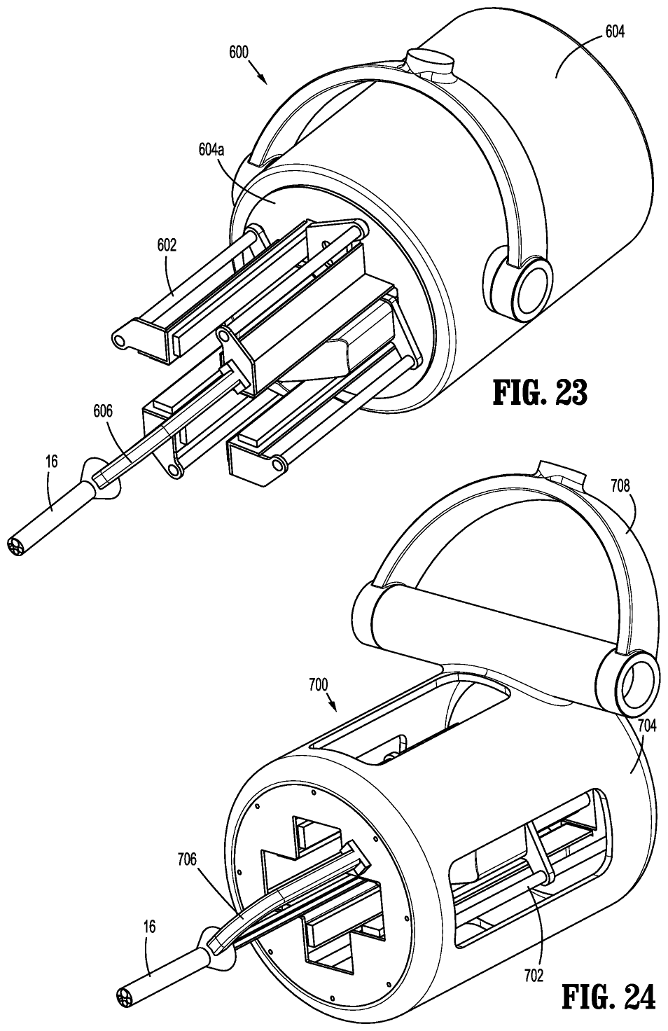

As seen in FIG. 23, yet another control drive assembly is generally shown as 600. Control drive assembly 600 is similar to control drive assembly 100, but includes a lead screw assembly 602 that is cantilevered to a distal end portion of a housing 604a of a control drive unit 604. A support arm 606 thereof can be cantilevered from the distal end portion of lead screw assembly 604 for supporting insertion tube 16 on a distal end portion thereof.

Alternatively, as seen in FIG. 24, one control drive assembly 700 includes a lead screw assembly 702 that is supported within a housing 704a of a control drive unit 704, on opposite ends of housing 704a, with support arm 706 extending distally from a distal end portion of housing 704a for supporting insertion tube 16. Control drive assembly 700 further includes a mounting clevis 708 supported on proximal end portion of housing 704a.

The disclosed structure can include any suitable mechanical, electrical, and/or chemical components for operating the disclosed system or components thereof. For instance, such electrical components can include, for example, any suitable electrical and/or electromechanical, and/or electrochemical circuitry, which may include or be coupled to one or more printed circuit boards. As appreciated, the disclosed computing devices (and/or servers) can include, for example, a “controller,” “processor,” “digital processing device” and like terms, and which are used to indicate a microprocessor or central processing unit (CPU). The CPU is the electronic circuitry within a computer that carries out the instructions of a computer program by performing the basic arithmetic, logical, control and input/output (I/O) operations specified by the instructions, and by way of non-limiting examples, include server computers. In some aspects, the controller includes an operating system configured to perform executable instructions. The operating system is, for example, software, including programs and data, which manages hardware of the disclosed apparatus and provides services for execution of applications for use with the disclosed apparatus. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. In some aspects, the operating system is provided by cloud computing.

In some aspects, the term “controller” may be used to indicate a device that controls the transfer of data from a computer or computing device to a peripheral or separate device and vice versa, and/or a mechanical and/or electromechanical device (e.g., a lever, knob, etc.) that mechanically operates and/or actuates a peripheral or separate device.

In aspects, the controller includes a storage and/or memory device. The storage and/or memory device is one or more physical apparatus used to store data or programs on a temporary or permanent basis. In some aspects, the controller includes volatile memory and requires power to maintain stored information. In various aspects, the controller includes non-volatile memory and retains stored information when it is not powered. In some aspects, the non-volatile memory includes flash memory. In certain aspects, the non-volatile memory includes dynamic random-access memory (DRAM). In some aspects, the non-volatile memory includes ferroelectric random-access memory (FRAM). In various aspects, the non-volatile memory includes phase-change random access memory (PRAM). In certain aspects, the controller is a storage device including, by way of non-limiting examples, CD-ROMs, DVDs, flash memory devices, magnetic disk drives, magnetic tapes drives, optical disk drives, and cloud-computing-based storage. In various aspects, the storage and/or memory device is a combination of devices such as those disclosed herein.

In various aspects, the memory can be random access memory, read-only memory, magnetic disk memory, solid state memory, optical disc memory, and/or another type of memory. In various aspects, the memory can be separate from the controller and can communicate with the processor through communication buses of a circuit board and/or through communication cables such as serial ATA cables or other types of cables. The memory includes computer-readable instructions that are executable by the processor to operate the controller. In various aspects, the controller may include a wireless network interface to communicate with other computers or a server. In aspects, a storage device may be used for storing data. In various aspects, the processor may be, for example, without limitation, a digital signal processor, a microprocessor, an ASIC, a graphics processing unit (“GPU”), field-programmable gate array (“FPGA”), or a central processing unit (“CPU”).

The memory stores suitable instructions and/or applications, to be executed by the processor, for receiving the sensed data (e.g., sensed data from camera), accessing storage device of the controller, generating a raw image based on the sensed data, comparing the raw image to a calibration data set, identifying an object based on the raw image compared to the calibration data set, transmitting object data to a post-processing unit, and displaying the object data to a graphic user interface. Although illustrated as part of the disclosed structure, it is also contemplated that a controller may be remote from the disclosed structure (e.g., on a remote server), and accessible by the disclosed structure via a wired or wireless connection. In aspects where the controller is remote, it is contemplated that the controller may be accessible by, and connected to, multiple structures and/or components of the disclosed system.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on the disclosed controllers or on a user device, including for example, on a mobile device, an IOT device, or a server system.

In some aspects, the controller includes a display to send visual information to a user. In various aspects, the display is a cathode ray tube (CRT). In various aspects, the display is a liquid crystal display (LCD). In certain aspects, the display is a thin film transistor liquid crystal display (TFT-LCD). In aspects, the display is an organic light emitting diode (OLED) display. In certain aspects, on OLED display is a passive-matrix OLED (PMOLED) or active-matrix OLED (AMOLED) display. In aspects, the display is a plasma display. In certain aspects, the display is a video projector. In various aspects, the display is interactive (e.g., having a touch screen) that can detect user interactions/gestures/responses and the like. In some aspects, the display is a combination of devices such as those disclosed herein.

The controller may include or be coupled to a server and/or a network. As used herein, the term “server” includes “computer server,” “central server,” “main server,” and like terms to indicate a computer or device on a network that manages the disclosed apparatus, components thereof, and/or resources thereof. As used herein, the term “network” can include any network technology including, for instance, a cellular data network, a wired network, a fiber-optic network, a satellite network, and/or an IEEE 802.11a/b/g/n/ac wireless network, among others.

In various aspects, the controller can be coupled to a mesh network. As used herein, a “mesh network” is a network topology in which each node relays data for the network. All mesh nodes cooperate in the distribution of data in the network. It can be applied to both wired and wireless networks. Wireless mesh networks can be considered a type of “Wireless ad hoc” network. Thus, wireless mesh networks are closely related to Mobile ad hoc networks (MANETs). Although MANETs are not restricted to a specific mesh network topology, Wireless ad hoc networks or MANETs can take any form of network topology. Mesh networks can relay messages using either a flooding technique or a routing technique. With routing, the message is propagated along a path by hopping from node to node until it reaches its destination. To ensure that all its paths are available, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. This concept can also apply to wired networks and to software interaction. A mesh network whose nodes are all connected to each other is a fully connected network.

In some aspects, the controller may include one or more modules. As used herein, the term “module” and like terms are used to indicate a self-contained hardware component of the central server, which in turn includes software modules. In software, a module is a part of a program. Programs are composed of one or more independently developed modules that are not combined until the program is linked. A single module can contain one or several routines, or sections of programs that perform a particular task.

As used herein, the controller includes software modules for managing various aspects and functions of the disclosed system or components thereof.

The disclosed structure may also utilize one or more controllers to receive various information and transform the received information to generate an output. The controller may include any type of computing device, computational circuit, or any type of processor or processing circuit capable of executing a series of instructions that are stored in memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, cause the one or more processors to perform one or more methods and/or algorithms.

The phrases “in an aspect,” “in aspects,” “in various aspects,” “in some aspects,” “in other aspects” or the like may each refer to one or more of the same or different aspects in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to all such states. Reference to a program may encompass the actual instructions and/or the intent of those instructions.

Securement of any of the components of the disclosed devices may be effectuated using known securement techniques such welding, crimping, gluing, fastening, etc.

Persons skilled in the art will understand that the structures and methods specifically described herein and shown in the accompanying figures are non-limiting exemplary aspects, and that the description, disclosure, and figures should be construed merely as exemplary of aspects. It is to be understood, therefore, that this disclosure is not limited to the precise aspects described, and that various other changes and modifications may be effectuated by one skilled in the art without departing from the scope or spirit of the disclosure. Additionally, the elements and features shown or described in connection with certain aspects may be combined with the elements and features of certain other aspects without departing from the scope of this disclosure, and that such modifications and variations are also included within the scope of this disclosure. Accordingly, the subject matter of this disclosure is not limited by what has been particularly shown and described.

What is claimed is:

1. A robotic surgical system, comprising:
an instrument cart having a setup arm assembly; and
a control drive assembly coupled to the instrument cart, the control drive assembly including:
at least one surgical instrument; and
a control drive unit that is secured to the setup arm assembly, the control drive unit movable relative to the setup arm assembly, the control drive unit including a housing and a motor block assembly supported within the housing, the housing including a front face, a rear face, and a sidewall extending between the front and rear faces, the motor block assembly including at least one motor block module that is selectively attachable to the at least one surgical instrument via front or side loading, the at least one motor block module being remotely actuatable to control the at least one surgical instrument, the at least one motor block module axially movable relative to the housing to facilitate movement of the at least one surgical instrument relative to the housing.

2. The robotic surgical system of claim 1, wherein the control drive unit includes a mounting clevis that secures the housing to the setup arm assembly.

3. The robotic surgical system of claim 2, wherein the mounting clevis is pinned to the sidewall of the housing to enable the housing to pitch relative to the mounting clevis.

4. The robotic surgical system of claim 3, wherein the mounting clevis is secured to the setup arm assembly by a yaw pin that enables the housing to yaw with the mounting clevis relative to the setup arm assembly.

5. The robotic surgical system of claim 1, wherein the at least one motor block module includes a plurality of motor block modules, each motor block module of the plurality of motor block modules is independently axially movable relative to the other motor block modules.

6. The robotic surgical system of claim 5, further comprising a support arm extending from the control drive unit, the support arm secured to an insertion tube on a distal end portion of support arm.

7. The robotic surgical system of claim 6, wherein the at least one surgical instrument includes a plurality of surgical instruments, and wherein the plurality of surgical instruments is secured to the plurality of motor blocks.

8. The robotic surgical system of claim 7, wherein the plurality of surgical instruments is selectively movable through the insertion tube.

9. The robotic surgical system of claim 1, wherein the at least one motor block module supports a sterile interface module that couples the at least one surgical instrument to the at least one motor block module.

10. The robotic surgical system of claim 1, wherein the at least one motor block module includes at least one motor that imparts rotational force to the at least one surgical instrument.

11. A surgical system, comprising:
a housing including a front face, a rear face, and a sidewall extending between the front and rear faces;
a support arm extending distally from the front face of the housing;
an insertion tube supported on a distal end portion of the support arm;
at least one surgical instrument; and
a motor block assembly supported within the housing, the motor block assembly including at least one motor block module that is selectively attachable to the at least one surgical instrument via front or side loading, the at least one motor block module being remotely actuatable to control the at least one surgical instrument, the at least one motor block module axially movable relative to the housing to facilitate movement of the at least one surgical instrument relative to the insertion tube.

12. The surgical system of claim 11, wherein the housing supports a mounting clevis for selectively securing the housing to an instrument cart.

13. The surgical system of claim 12, wherein the mounting clevis is pinned to the sidewall of the housing to enable the housing to pitch relative to the mounting clevis.

14. The surgical system of claim 13, wherein the mounting clevis is selectively securable to a setup arm assembly of the instrument cart by a yaw pin that enables the housing to yaw with the mounting clevis relative to the setup arm assembly.

15. The surgical system of claim 11, wherein the at least one motor block module includes a plurality of motor block modules, each motor block module of the plurality of motor block modules is independently axially movable relative to the other motors block modules.

16. The surgical system of claim 15, wherein the at least one surgical instrument includes a plurality of surgical instruments, and wherein the plurality of surgical instruments is secured to the plurality of motor block modules.

17. The surgical system of claim 16, wherein the plurality of surgical instruments is selectively movable through the insertion tube.

18. The surgical system of claim 11, wherein the at least one motor block module supports a sterile interface module that couples the at least one surgical instrument to the at least one motor block module.

19. The surgical system of claim 11, wherein the at least one motor block module includes at least one motor that imparts rotational force to the at least one surgical instrument.

20. A control unit assembly for a robotic surgical system, the control unit assembly including:
a housing including a front face, a rear face, and a sidewall extending between the front and rear faces;
at least one surgical instrument; and
a motor block assembly supported within the housing, the motor block assembly including at least one motor block module that is selectively attachable to the at least one surgical instrument via front or side loading, the at least one motor block module being remotely actuatable to control the at least one surgical instrument, the at least one motor block module axially movable relative to the housing to facilitate movement of the at least one surgical instrument relative to the housing.
